(19) United States
(12) Patent Application Publication
(10) Pub. No.: US 2007/0027119 A1
(43) Pub. Date: Feb. 1, 2007

(54) Antibacterial composition and method of use

(76) Inventors: Fahim U. Ahmed, Greensboro, NC (US); N. Camelia Traistaru, Gladstone, MO (US); Thomas C. Hemling, Village of Loch LLoyd, MO (US)

Correspondence Address: LATHROP & GAGE LC, 2345 GRAND AVENUE, SUITE 2800, KANSAS CITY, MO, 64108, US

(21) Appl. No.: 11/192,571

(22) Filed: Jul. 29, 2005

(51) Int. Cl. A61K 31/60; A61K 31/192; A61K 31/198; A61K 31/19; A61K 31/20; A61K 31/045

(52) U.S. Cl. 514/159; 514/557; 514/560; 514/574; 514/561; 514/568; 514/563; 514/724; 514/730

(57) ABSTRACT

Use of a liquid composition comprising at least two agents selected from lactic acid, salicylic acid, benzyl alcohol, and/or low molecular weight aliphatic alcohol having less than five carbon atoms for the manufacture of an antimicrobial solution is disclosed. In a preferred embodiment, the antimicrobial solution is formulated as a teat dip for lactating animals, particularly cows. The composition may be used as a prophylactic treatment or for wound healing, and is particularly useful against mastitis. The antimicrobial compositions may be used in personal care, hard surface care including hard surface disinfection in households, food processing, hospitals, restaurants, hotels, showers, or topically as hand soaps, surgical scrubs, and hoof disease mitigators.

### BACKGROUND

### Field of the Invention

The present invention pertains to the field of topical biocidal liquid compositions of the type that may be used to control or destroy pathogenic microorganisms. More particularly, various antimicrobial agents are shown to work with cooperative effects against microorganisms in a wide variety of applications.

Antimicrobial compositions may be used to reduce the risk of infection. These materials are known and regularly used for hard surface disinfection in hospitals, lavatories, food preparation facilities and offices. Other uses include the control of pathogenic organisms on skin surfaces. The compositions may be used on human skin, for example, to reduce the transmission of disease or infection, as surgical scrub solutions or hand sanitizers. The compositions may be used in veterinary applications for the control or prevention of hoof diseases and mastitis. Prevention of mastitis is also a major goal of the dairy industry. Contact of the bovine or ovine mammary gland with pathogenic microorganisms, usually bacteria but occasionally yeast or fungi, can result in the disease of mastitis.

Mastitis is the single most costly disease affecting the dairy industry. Annual economic losses due to mastitis approximate $185 per dairy animal. This totals to approximately $1.7 billion annually for the entire United States market. Mastitis is always a potentially serious infection. Severe cases may cause death to the dairy animal. Milder cases are more common, but may have serious consequences, such as long term damage to the animal, loss of milk production for the dairy farmer and an unacceptable increase in veterinary costs.

To reduce mastitis, commercial teat dips have been developed which are usually administered to the teat by dipping or spraying the teat prior to milking as well as after removal of the milking cup. Teat dips applied subsequent to milking may form a thick composition, film or barrier that remains on the teat until the next milking, which is generally 8 to 12 hours later.

Commercially available teat dips may be divided into two primary classifications, namely, non-barrier and barrier dips. The non-barrier teat dips are strictly antimicrobial and are applied to kill microorganisms that are already present in the teat canal or on the surface of the teat skin. By design, the microbiological effect is substantially immediate, targeting the contagious organisms that may be transferred between animals during the pre-milking, milking and post milking process. The barrier dips may also be antimicrobial and are applied to form a prophylactic film or coating that may prevent microbes from contacting the teat.

Teat dips have used a variety of antimicrobial agents. U.S. Pat. No. 2,739,922 issued to Shelanski describes the use of polymeric N-vinyl pyrrolidone in combination with iodophors. U.S. Pat. No. 3,993,777 issued to Caughman et al. describes the use of halogenated quaternary ammonium compounds. U.S. Pat. No. 4,199,602 issued to Lentsch describes the use of iodophors, chlorine releasing compounds (e.g. alkali hypochlorite), oxidizing compounds (e.g. hydrogen peroxide, peracids), protonated carboxylic acids (e.g. heptanoic, octanoic, nonanoic, decanoic, undecanoic acids), and nitroalkanols. U.S. Pat. No. 4,434,181 issued to Marks, Sr. et al. describes the use of acid anionics (e.g. alkylaryl sulfonic acids), chlorine dioxide (from alkali chlorite), and bisbiguanides such as chlorhexidine.

Some of the available teat dip agents suffer from serious drawbacks. For example, iodine, hypochlorite, chlorine dioxide, and hypochlorous acid are powerful disinfectants and strong oxidants, but they are also particularly noxious for both humans and animals. Additionally, the use of overly powerful disinfectants may contribute to the mastitis problem by causing irritation of the teat skin, thus providing an opportunistic site which promotes infection. The Lentsch '602 patent recognizes that iodophors and such chlorine-based biocides as hypochlorite, chlorine dioxide, and hypochlorous acid have achieved the widest commercial acceptance; however, teat dips of the future may have to be iodine-free. Furthermore, the iodine-based and chlorine-based compositions may induce sensitized reactions in cow teats. Other biocides including chlorhexidine have become the focus of regulatory concern. On the other hand, less powerful teat dip agents, such as fatty acids and anionic surfactants, are often not broad enough in their antibacterial spectrum to provide complete germicidal protection.

From a consumption point of view, it is known that relatively small quantities of iodine and chlorhexidine can result in taste changes of the milk as well as problems in the manufacture of dairy products. Furthermore, milk products must meet food and drug regulations which take into consideration ingestion of residual teat dip agents. There may be concern, for example, about increased iodine consumption because iodine is linked to thyroid function and it is recommended that some populations, such as pregnant women, limit their intake. Also, iodine associates with problems of staining, and some operators/users develop allergic symptoms such as skin irritation and sensitization from iodine-based product use.

Thus, although many teat dip products are available, there is a continuing need for antimicrobial compositions that are highly effective, yet mild to skin and less noxious, irritating or dangerous in a wide range of applications, such as teat dipping, skin cleansing, and hard surface disinfection.

### SUMMARY

The present instrumentalities overcome the problems outlined above and advance the art by providing highly effective but substantially nonirritating antimicrobial liquid compositions that may be formulated without halogen-derived antimicrobial active agents. The antimicrobial compositions provide a substantial reduction in Gram positive and Gram negative bacteria. This type of substantial reduction may, for example, be on the order of a three or four log reduction or a substantially complete kill that is greater than a 99.999999% reduction.

The compositions reported herein involve the discovery, hitherto unreported, that substantially higher antimicrobial protection can be obtained when an organic acid is combined with benzyl alcohol and/or a low molecular weight aliphatic alcohol moiety having a carbon number less than five. Preferred low molecular weight aliphatic alcohols have from one to three carbon atoms. Especially preferred alcohols include benzyl alcohol, ethyl alcohol, n-propyl alcohol, and isopropyl alcohol. The aliphatic alcohol may be also be selected from substituted alcohol wherein the substituent(s) moiety is an aromatic such as phenyl group, e.g. phenyl alkyl alcohol (phenyl alkanol). Phenyl alkyl alcohol (phenyl alkanol) can be chosen, wherein alkyl group is selected from methyl, ethyl, isopropyl, n-propyl, n-butyl, isobutyl, ethyledene, propyledene moiety. In one example, an unexpected efficacy is achieved when various combinations of lactic acid, salicylic acid, and benzyl alcohol, or isopropyl alcohol are combined with a carrier to provide a single antimicrobial formulation.

As used herein, the term “organic acid” means an organic compound that is an acid. The most common examples are the carboxylic acids having an acidity that derives from a carboxyl group —COOH. Other groups may also impart weak acidity, especially hydroxyl (—OH) groups, thiol (—SH) groups, enols, —OSO₃H groups, and phenols. Preferred organic acids have a carbon number less than twenty, and this number is even more preferably less than ten. The organic acids may be aliphatic, aromatic, unsubstituted or substituted with other functional groups for example, such as hydroxyl. The substituent(s) may be attached to any position of the carbon chain or onto the aromatic ring. The organic acids may, for example, be lactic acid, salicylic acid, tartaric acid, citric acid, glycolic acid, ascorbic acid, maleic acid, succinic acid, mandelic acid, dodecylbenzenesulfonic acid, propionic acid, gluconic acid, malic acid, benzoic acid, aspartic acid, acetic acid, oxalic acid, glutamic acid, adipic acid, hexanoic acid, octanoic acid, nonanoic acid, decanoic acid, undecanoic acid and combinations thereof. In another aspect, inorganic acids having pKₐ characteristics approximating those of organic acids may also be used. In one such example, sulfamic acid may be used. Particularly preferred organic acids include lactic acid, salicylic acid and combinations thereof.

In one aspect, the disclosure provides a method and a composition for the treatment or prevention of infection in a subject. The method includes administering one or more organic acids mixed with benzyl alcohol and/or a low molecular weight aliphatic alcohol moiety having less than five carbon atoms. The method comprises administering to a subject a therapeutically effective amount of a topical composition that contains these antimicrobial agents.

Those skilled in the art will appreciate additional objects and advantages in the detailed description below. All references specifically disclosed in this specification are hereby incorporated by reference.

### DETAILED DESCRIPTION

There will now be shown and described as a particular embodiment, an antimicrobial liquid composition that contains an organic acid mixed with benzyl alcohol and/or a low molecular weight aliphatic alcohol having less than five carbon atoms. These ingredients may be mixed with a carrier that is formulated according to the intended environment of use.

In one aspect, the antimicrobial composition may contain at least two antimicrobial agents selected from organic acids, such as lactic acid and/or salicylic acid, and benzyl alcohol, or isopropyl alcohol in a pharmaceutically acceptable carrier. A pharmaceutically acceptable carrier may, for example, be water.

The carrier may include, for example, an additive selected from a buffering agent, an emollient, a humectant, a preservative, a barrier forming agent, a surfactant or wetting agent, a viscosity control agent, a colorant, an opacifying agent, and any combinations thereof.

A broader object of the disclosed instrumentalities is to provide a biocidal composition that may be used, for example, according to any purpose for antibacterial or bactericidal properties. In a particular embodiment, the composition is intended to be used as a teat dip. In another particular embodiment, the composition is intended to be used as a hand sanitizer, a skin cleanser, a surgical scrub, a wound care agent, a disinfectant, a mouthwash, bath/shower gel, a hard surface sanitizer and the like. Preferred compositions for skin applications have a pH of about 2.5 to about 7.5 and provide a substantial reduction, e.g., greater than 99.999999%, in Gram positive and Gram negative bacterial populations.

Another object is to provide a composition which, when applied, results in a wound healing effect. The composition assists in a faster and qualitatively improved healing of wounds by decreasing the number of microorganisms in the vicinity of the wound.

Yet another object is to provide a composition which, when applied, results in a prophylactic action against mastitis.

Methods of preparing compositions may involve dissolving a desired concentration of antimicrobial agents and, optionally, any desired additives in a selected pharmaceutical carrier. The solution is then mixed, for example in a mixer, to form a final antimicrobial composition. Useful concentrations are those where the percentage of each antimicrobial agent by total weight of the composition is preferably from about 0.02 to 20% by weight of the composition, and a pharmaceutical carrier may be present form 80 to 99.98% by weight. More preferably, this is from about 0.03 to 15% of each antimicrobial agent and from about 85 to 99.97% of a pharmaceutical carrier. Most preferably, this is from about 0.04 to 10% of each antimicrobial agent and from about 90 to 99.96% of a pharmaceutical carrier. Still more preferably, this is from about 0.05 to 8% of each antimicrobial agent and from about 92 to 99.95% of a pharmaceutical carrier.

Useful concentrations are those where the percentage of each functional ingredient or mixture of ingredients including antibacterial agents by total weight of the composition is preferably from about 0.02 to 30% of each antimicrobial agent and 70 to 99.98% of a pharmaceutical carrier; more preferably from about 0.03 to 25% of each antimicrobial agent and from about 75 to 99.97% of a pharmaceutical carrier; and most preferably from about 0.04 to 20% of each antimicrobial agent and from about 80 to 99.96% of a pharmaceutical carrier, and still more preferably from about 0.05 to 15% of each antibacterial agent and from about 85 to 99.95% of a pharmaceutical carrier.

As used herein, the term “subject” shall include humans and terrestrial animals. For example, the subject can be a domestic livestock species, a laboratory animal species, a zoo animal, a companion animal or a human. In a particular embodiment, “subject” refers more specifically to any lactating animal; preferably, the subject is a cow.

The phrase “therapeutically effective amount” is intended to qualify the amount of the topical composition which will achieve the goal of decreased microbial concentration. “Therapeutically effective” may also refer to improvement in disorder severity or the frequency of incidence over no treatment.

The term “topical” shall refer to any composition applied to the epidermis. Topical shall also refer to compositions used as mouthwashes.

The term “additive” shall mean any component that is not an antimicrobial agent or a pharmaceutical carrier. A pharmaceutical carrier is generally a bulk solvent used to dilute or solubilize the components of the composition.

The terms “teat dip” or “teat dipping” shall be interpreted broadly and in accordance with the terminology used in the art of dairy farming. Thus, the composition is not only intended for dipping of the teats but it can, of course, be applied in other ways, such as by spraying, but still fall within the recognized terms teat dip or teat dipping composition or agent.

As used herein unless other wise specified, the term “antimicrobial” describes am biocidal effect that may be, for example, an antibacterial, antifungal, antiviral, bacteriostatic, disinfecting, or sanitizing effect.

As shown in the examples below, combinations of the antimicrobial agents may include an organic acid with benzyl alcohol and/or a low molecular weight aliphatic alcohol having a carbon number less than five. In particular, lactic acid, salicylic acid, benzyl alcohol, and/or isopropyl alcohol may suffice to make effective biocidal compositions. These basic ingredients may be formulated using additional antimicrobial agents, barrier-forming agents, viscosity control agents, pH adjusting agents, wetting agents, opacifying agents, and carriers to make a wide variety of products.

Additional Antimicrobial Agents

Traditional antimicrobial agents are the components of a composition that destroy microorganisms or prevent or inhibit their replication. In one aspect, the combined antimicrobial agents discussed above may be used to replace or eliminate the need for traditional antimicrobial agents in a wide variety of applications. In another aspect, antimicrobial compositions according to the disclosed embodiments below may be used in combination with these traditional antimicrobial agents, for example, to achieve an effective kill at lower concentrations of traditional antimicrobial agents.

Traditional antimicrobial agents include iodophors, quaternary ammonium compounds, hypochlorite releasing compounds (e.g. alkali hypochlorite, hypochlorous acid), oxidizing compounds (e.g. hydrogen peroxide, peracids; hypochlorite), protonated carboxylic acids (e.g. heptanoic, octanoic, nonanoic, decanoic, undecanoic acids), acid anionics (e.g. alkylaryl sulfonic acids), chlorine dioxide from alkali chlorite by an acid activator, and bisbiguanides such as chlorhexidine. Phenolic antibacterial agents may be chosen from 2,4,4′-trichloro-2′-hydroxydiphenylether, which is known commercially as triclosan and may be purchased from Ciba Specialty Chemicals as IRGASAN™ and IRGASAN DP 300™) having the following structural formula (I):

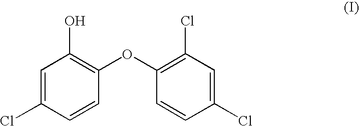

Another such antibacterial agent is 4-chloro-3,5-dimethyl phenol, which is also known as PCMX and is commercially available as NIPACIDE PX and NIPACIDE PX-P having the following structural formula (II):

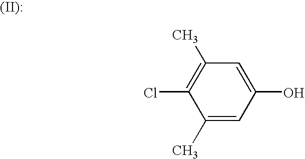

Other traditional germicides include formaldehyde releasing compounds such as glutaraldehyde, 2-bromo-2-nitro-1,3-propanediol (Bronopol) having the following structural formula (III). 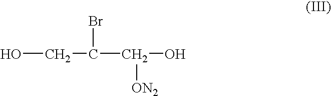
Barrier Forming Agents

Barrier and film forming agents are those components of a teat dipping composition that remain in contact with the teat between milking cycles. Barrier and film forming agents coat the teat skin and, optionally, the udder. Barrier agents may form a plug at the end of the open teat canal. Typical barrier and film forming agents include thick creams or emollients (made with viscosity control agents), films, polymers, latex and the like. Some nonionic surfactants may help further enhance the barrier property in addition to wetting properties. Examples of such surfactants may include, without limitation, Pluronic P105 and Pluronic F108. A latex material that provides an effective covering of the teat is described in U.S. Pat. No. 4,113,854. Suitable barrier forming agents include, for example, latex, arabinoxylanes, glucomannanes, guar gum, johannistree gums, cellulose, methyl cellulose, ethyl cellulose, hydroxyethyl cellulose, hydroxymethyl cellulose, carboxyethyl cellulose, carboxymethyl cellulose, starch, hydroxyethyl starch, gum arabic, curdlan, pullulan, dextran, polysulfonic acid, polyacryl amide, high molecular weight polyacrylate, high molecular weight cross-linked polyacrylate, carbomer, glycerol, sodium alginate, sodium alginate cross-linked with calcium salt, xanthan gum, poly(vinyl alcohol) (PVA) and poly(N-vinylpyrrolidone) (PVP). Preferred barrier-forming agents include xanthan gum, carboxymethyl cellulose, sodium alginate, sodium alginate cross-linked with calcium salt, PVA, hydroxyethyl cellulose and PVP.

Viscosity Control Agents

Viscosity control agents may be added to formulate the antimicrobial applications according to an intended environment of use. In one example, it is advantageous for some formulations to have an optimized solution viscosity to impart vertical clinging of the product onto a teat. This type of viscous product, especially one having a suitable thixotropic, pseudoplastic or viscoelastic gel strength, minimizes dripping of the product to avoid wastage and is particularly advantageous in teat dip formulations. Teat dip formulations may benefit from a preferred dynamic viscosity ranging from 100 cPs to 3000 cPs. Other applications including hard surface disinfectants have a preferred dynamic viscosity ranging from about 1 cPs to 300 cPs. In another example, the amount of viscosity control agents may be substantially reduced or even eliminated in other formulations, such as surface or floor disinfectants where easy cleanup is desired. An intermediate or medium viscosity composition may be useful in a hand cleaner or personal care product. It is seen from these examples that the antimicrobial compositions may be formulated for a wide variety of applications by altering the amount of viscosity control agents.

Suitable viscosity control agents include hemicellulose, for example arabinoxylanes and glucomannanes; plant gum materials, for example guar gum and johannistree gums; cellulose and derivatives thereof, for example methyl cellulose, ethyl cellulose, hydroxypropyl cellulose, hydroxyethyl cellulose or carboxymethyl cellulose; starch and starch derivatives, for example hydroxyethyl starch or cross linked starch; microbial polysaccharides, for example xanthan gum, sea weed polysaccharides, for example sodium alginate, carrageenan, curdlan, pullulan or dextran, dextran sulfate, whey, gelatin, chitosan, chitosan derivatives, polysulfonic acids and their salts, polyacrylamide, and glycerol. Preferred viscosity controlling agents are, different types of cellulose and derivatives thereof, particularly hydroxyalkyl cellulose, methyl cellulose, and glycerol. High molecular weight (MW>1,000,000) cross-linked polyacrylic acid type thickening agents are the products sold by B.F. Goodrich (now Noveon) under their Carbopol® trademark, especially Carbopol® 941, which is the most ion-insensitive of this class of polymers, and Carbopol® 940 and Carbopol®934. The Carbopol® resins, also known as “Carbomer”, are hydrophilic high molecular weight, cross-linked acrylic acid polymers having an average equivalent weight of 76, and the general structure illustrated by the following formula (IV) as reported in U.S. Pat. No. 5,225,096: 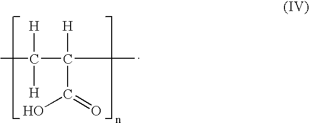
Carbopol® 941 has a molecular weight of about 1,250,000, Carbopol® 940 has a molecular weight of approximately 4,000,000 and Carbopol 934 has a molecular weight of approximately 3,000,000. The Carbopol® resins are cross-linked with polyalkenyl polyether, e.g. about 1% of a polyallyl ether of sucrose having an average of about 5.8 allyl groups for each molecule of sucrose. Further detailed information on the Carbopol® resins is available from B.F. Goodrich (Noveon), see for example, the B.F. Goodrich catalog GC-67, Carbopol® Water Soluble Resins. Clays and modified clays such as bentonite or laponite can also be used as thickeners. Cothickeners are often added to improve the stability of the gel matrix, for example, colloidal alumina or silica, fatty acids or their salts. A latex material that provides an effective covering of the teat is described in U.S. Pat. No. 4,113,854. Typical film forming ingredients include xanthan gum, carboxymethyl cellulose, sodium alginate, sodium alginate cross-linked with calcium salt, polysulfonic acids and their salts, polyacrylamide, poly(vinyl alcohol) (PVA), hydroxyethyl cellulose and poly(N-vinylpyrrolidone) (PVP).
pH Adjusting Agents

A composition pH value may be selectively adjusted by the addition of acidic or basic materials. Generally, an acidic pH is preferred. Suitable acids for use as pH adjusting agents may include, for example, citric acid, phosphoric, phosphorous, sulfamic, nitric, and hydrochloric acids. Mineral acids may be used to drastically lower the pH. The pH may be raised or made more alkaline by addition of an alkaline agent such as sodium hydroxide, potassium hydroxide, sodium carbonate, or sodium bicarbonate or combinations there of.

The physical property of pH may be adjusted by acid or base addition, and is broadly preferred in the range of from 2.5 to 7.0 for use in teat dip formulations and other formulations that are intended to contact the skin. In a still more preferred sense this is from 3.25 to 5.0. Hard surface and commercial disinfectants may be provided with still lower pH values, such as 1.0 or 0.5.

Wetting Agents and Surfactants

Wetting agent(s) or surface active agent(s) are also known as surfactants. These materials may be included to formulate the antimicrobial compositions for an intended environment of use. Typical wetting agents and surfactants are used to wet the surface of application, reduce surface tension of the surface of application so that the product can penetrate easily on the surface and remove unwanted soil. The wetting agents or surfactants of the formulation increase overall detergency of the formula, solubilize or emulsify some of the organic ingredients that otherwise would not dissolve or emulsify, and facilitate penetration of active ingredients deep onto the surface of the intended application surfaces, such as teat skins and teats.

Suitably effective surfactants may include anionic, cationic, nonionic, zwitterionic and amphoteric surfactants. Wetting agents and surfactants used in the inventive applications can be high foaming, low foaming and non foaming type. Suitable anionic surfactants can be chosen from a linear alkyl benzene sulfonic acid, a linear alkyl benzene sulfonate, an alkyl α-sulfomethyl ester, an α-olefin sulfonate, an alcohol ether sulfate, an alkyl sulfate, an alkylsulfo succinate, a dialkylsulfo succinate, and alkali metal, alkaline earth metal, amine and ammonium salts thereof. Specific examples are linear C₁₀-C₁₆ alkyl benzene sulfonic acid, linear C₁₀-C₁₆ alkyl benzene sulfonate or alkali metal, alkaline earth metal, amine and ammonium salt thereof e.g. sodium dodecylbenzene sulfonate, sodium C₁₄-C₁₆ α-olefin sulfonate, C₁₂-C₁₈ sodium methyl α-sulfomethyl ester and C₁₂-C_{18,} disodium methyl α-sulfo fatty acid salt. Suitable nonionic surfactants can be chosen from an alkyl polyglucoside, an alkyl ethoxylated alcohol, an alkyl propoxylated alcohol, an ethoxylatedpropoxylated alcohol, sorbitan, sorbitan ester, an alkanol amide. Specific examples include C₈-C₁₆ alkyl polyglucoside with a degree of polymerization ranging from 1 to 3 e.g., C₈-C₁₀ alkyl polyglucoside with a degree of polymerization of 1.5 (Glucopon® 200), C₈-C₁₆ alkyl polyglucoside with a degree of polymerization of 1.45 (Glucopon® 425), C₁₂-C₁₆ alkyl polyglucoside with a degree of polymerization of 1.6 (Glucopon® 625), and polyethoxylated polyoxypropylene block copolymers (poloxamers) including by way of example the Pluronic® poloxamers commercialized by BASF Chemical Co. Amphoteric surfactants can be chosen from alkyl betaines and alkyl amphoacetates. Suitable betaines include cocoamidopropyl betaine, and suitable amphoacetates include sodium cocoamphoacetate, sodium lauroamphoacetate and sodium cocoamphodiacetate.

Opacifying Agents and Dyes

An opacifying agent or dye is optionally included in the teat dip formulations. Color on the teat tells farmers that they did indeed dip a particular cow. To preclude any problems with possible contamination of milk, it is preferred that only FD&C Certified (food grade) dyes be used. There are many FD&C dyes available which are FD&C Red #40, FD&C Yellow #6, FD&C Yellow #5, FD&C Green #3 and FD&C Blue #1. Dyes used either alone or in combination are preferred. D&C Orange #4 can also be used. Titanium dioxide (TiO₂) is widely used as an opacifier and can also be used in combination with various colorants.

Preservatives

Some known teat dips and hand sanitizers include ethylenediaminetetraacetic acid (EDTA) and its alkali salts which can act as a chelating agent to remove metal ions from hard water. The metal ions, if not removed from the composition, serve as reaction sites for enzymes within the bacteria; the metalloenzyme reactions produce energy for bacterial cell replication. Other traditional preservatives but not limited to that are widely used, for example, paraban, methyl paraban, ethyl paraban, glutaraldehyde etc.

Pharmaceutical Carriers

A typical carrier or matrix for an antimicrobial composition is purified water, although one skilled in the art will readily understand that other solvents or compatible materials other than water may be used to achieve the effective amounts of germicidal agents. In some embodiments, a composition may contain at least about 70% water and preferably at least about 75% water by weight based on the total weight of the formulation. Propylene glycol can also be used as a carrier either alone or in combination with water.

### EXAMPLES

The invention will now be further illustrated by the following non-limiting examples.

### Example 1

### Test of Antimicrobial Activity

Various standardized test methods are in place for the conduct of comparative tests regarding the efficacy of antimicrobial agents. The preferred standard is defined as AOAC Official Method 960.09, as published by the Association of Analytical Chemists (AOAC International) in 2000. Europeans tend to use other standards for this same purpose, such as the EN1040 and EN1656 test methods. All of these standards are incorporated by reference to the same extent as though fully disclosed herein.

According to the test methodology of AOAC 960.09, vertical agar plates were each covered with one of the solutions 1-36 that are described below in Tables 1-7. The vertical agar plates were then dipped in concentrated bacteria solutions. The agar plates were incubated in a heating cabinet overnight and the number of bacteria colonies were calculated and recorded as cfu/mL of the bacteria solution. Bacterial counts were determined by UV-Visible spectrophotometer. Controls were established as untreated vertical agar plates exposed only to bacterial solutions.

Materials and Reagents

The test bacteria were Eschericia coli (ATCC 11229), which were originally isolated from mastitis infection and obtained on commercial order from Mastitlaboratoriet, SVA; and Staphococcus aureus (ATCC6538 from Remel, Inc. of Kansas City) which was also isolated from mastitis infection.

Procedure

E. coli and S. aureus were sterile transferred (sterile bench) from storage agar to separate sterilized trypton soy broth and incubated overnight at 37° C. in a shake incubator. The following morning the bacteria were washed three times with a physiological salt solution. The optical density at 650 nm was measured with a spectrophotometer and, by means of standard curves, the bacteria density was adjusted.

Bacteria Tests

Each bacterial type was treated separately according to the following procedure:

Two agar plates were lowered into a solution selected from solutions 1-36 as shown in Tables 1-7. Then the two treated agar plates and a third control plate were subjected to a bacteria solution of either E. coli or S. aureus. The agar plates were incubated overnight in a heat cabinet at 37° C. Following incubation, the plates and the number of colonies were inspected, averaged and registered.

Results

Shown below are the results of experiments carried out to determine the efficacy of various antimicrobial compositions against E. coli and S. aureus. The antimicrobial agents in the compositions were selected from organic acids, such as lactic acid and salicylic acid, benzyl alcohol, and isopropyl alcohol in any combination. Table 1 shows that when each of the antimicrobial agents was used individually, there was no effect on E. coli or S. aureus relative to the control. Table 2 shows that concentrations of individual components may be increased to a point where there is antimicrobial efficacy, but at the cost of composition stability where at these higher concentrations the solutions become unstable as indicated by notation indicating that the solutions have become hazy or have formed precipitates (ppt).

TABLE 1
Kill Property Study: Selection of Germicidals: One Germicide

| | Solution: | | | |
|---|---|---|---|---|
| | 1 | 2 | 3 | 4 |
| Antimicrobial Agents | | | | |
| Lactic Acid (88%) - 4.00% | + | | | |
| Salicylic Acid - 0.50% | | + | | |
| Benzyl Alcohol - 1.00% | | | + | |
| Isopropyl Alcohol - 3.00% | | | | + |
| Ingredients (wt %) | | | | |
| Water | 92.15 | 96.40 | 95.15 | 93.15 |
| Xanthan Gum (Novaxan D/ADM) | 0.40 | 0.40 | 0.40 | 0.40 |
| Polyvinylpyrrolidone K-30 | 0.80 | 0.80 | 0.80 | 0.80 |
| Salicylic Acid | | 0.50 | | |
| Allantoin | | | | |
| Isopropyl Alcohol | | | | 3.00 |
| L(+)-Lactic Acid (88%) | 4.00 | | | |
| USP-ADM | | | | |
| Benzyl Alcohol | | | 1.00 | |
| PEG-7-Glyceryl Cocoate | 0.50 | 0.50 | 0.50 | 0.50 |
| (Cetiol HE) | | | | |
| EO/PO/EO Block Copolymer | 0.50 | 0.50 | 0.50 | 0.50 |
| (Pluronic P105) | | | | |
| Sodium Dioctylsulfosuccinate | 0.15 | 0.15 | 0.15 | 0.15 |
| (75%) | | | | |
| POE-20 Sorbitan Monooleate | 0.50 | 0.50 | 0.50 | 0.50 |
| (Polysorbate 80) (Tween 80) | | | | |
| Sodium Hydroxide (50%) | 1.00 | 0.25 | | |
| Phosphoric acid | | | 1.00 | 1.00 |
| pH (neat) | 3.52 | 3.49 | 3.49 | 3.60 |
| AOAC Germicidal Test 960.09 | | | | |
| (20° C./30 Sec) | | | | |
| E. Coli (Log Reduction) | No Kill | No Kill | No Kill | No Kill |
| Staph. Aureus (Log Reduction) | No Kill | No Kill | No Kill | No Kill |

TABLE 2
Kill Property Study: Selection of Germicidals: One Germicide-Higher Concentration

| | Solution | | | | | | | | |
|---|---|---|---|---|---|---|---|---|---|
| | 5 | 6 | 7 | 8 | 9 | 10 | 11 | 12 | 13 |
| Ingredients (wt %) | | | | | | | | | |
| Water | 94.40 | 89.15 | 86.15 | 94.90 | 93.90 | 92.90 | 95.90 | 95.15 | 94.40 |
| Xanthan Gum (Novaxan D/ADM) | 0.40 | 0.40 | 0.40 | 0.40 | 0.40 | 0.40 | 0.40 | 0.40 | 0.40 |
| Polyvinylpyrrolidone K-30 | 0.80 | 0.80 | 0.80 | 0.80 | 0.80 | 0.80 | 0.80 | 0.80 | 0.80 |
| Salicylic Acid | | | | | | | 1.00 | 1.50 | 2.00 |
| Allantoin | | | | | | | | | |
| L(+)-Lactic Acid (88%) USP-ADM | 2.00 | 6.00 | 8.00 | | | | | | |
| Benzyl Alcohol | | | | 2.00 | 3.00 | 4.00 | | | |
| PEG-7-Glyceryl Cocoate (Cetiol HE) | 0.50 | 0.50 | 0.50 | 0.50 | 0.50 | 0.50 | 0.50 | 0.50 | 0.50 |
| EO/PO/EO Block Copolymer (Pluronic P105) | 0.50 | 0.50 | 0.50 | 0.50 | 0.50 | 0.50 | 0.50 | 0.50 | 0.50 |
| Sodium Dioctylsulfosuccinate(75%) | 0.15 | 0.15 | 0.15 | 0.15 | 0.15 | 0.15 | 0.15 | 0.15 | 0.15 |
| Polysorbate 80 (Tween 80) | 0.50 | 0.50 | 0.50 | 0.50 | 0.50 | 0.50 | 0.50 | 0.50 | 0.50 |
| Sodium Hydroxide (50%) | 0.75 | 2.00 | 3.00 | | | | 0.25 | 0.50 | 0.75 |
| Phosphoric acid* | | | | 0.25 | 0.25 | 0.25 | | | |
| pH (Neat) | 3.56 | 3.50 | 3.52 | 3.53 | 3.51 | 3.49 | 3.49 | 3.49 | 3.50 |
| AOAC Germicidal Test 960.09 (20° C./30 Sec) | | | | | | | | | |
| E. Coli (Log Reduction) | No Kill | 0.9 | 1.64 | 4.74 | 8.00 | 8.00 | 8.00 | 8.00 | 8.00 |
| Staph. Aureus (Log Reduction) | No Kill | No Kill | No Kill | 4.20 | 7.80 | 7.80 | 7.80 | 7.80 | 7.80 |
| Stability after 2 days | OK | OK | OK | OK | Hazy | Hazy | Precipitate | Precipitate | Precipitate |

*Phosphoric Acid was used to adjust the pH of the formula; all formula were adjusted to have a pH close to 3.50

The results of Table 1 show that individual active ingredients 4% (w/w) lactic acid, 1% benzyl alcohol, 0.5% salicylic acid, and 3% isopropyl alcohol, had virtually no quantifiable killing effect. Table 2 shows that these ingredients may be increased on an individual basis to a point where some killing effect on the order of one to two logs is observed against E. coli, but not S. aureus (solutions 5-7). Further increases of the antimicrobial concentration are infeasible because the solutions become unstable after two days, as indicated by haziness and the formation of precipitates.

As shown in Table 3, all solutions containing two of the antimicrobial agents that were used individually according to Table 1 showed some efficacy against at least one strain of bacteria. The best results were obtained with solution 15 which contained 4% lactic acid and 1% benzyl alcohol.

TABLE 3
Kill Property Study: Selection of Germicidals: Two Germicides
Combined

| | Solution: | | | | | |
|---|---|---|---|---|---|---|
| | 14 | 15 | 16 | 17 | 18 | 19 |
| Antimicrobial Agents | | | | | | |
| Lactic Acid (88%) - 4.00% | + | + | + | | | |
| Salicylic Acid - 0.50% | + | | | + | + | |
| Benzyl Alcohol - 1.00% | | + | | + | | + |
| Isopropyl Alcohol - 3.00% | | | + | | + | + |
| Ingredients (wt %) | | | | | | |
| Water | 91.40 | 91.15 | 89.15 | 95.40 | 93.40 | 92.65 |
| Xanthan Gum (Novaxan D/ADM) | 0.40 | 0.40 | 0.40 | 0.40 | 0.40 | 0.40 |
| Polyvinylpyrrolidone K-30 | 0.80 | 0.80 | 0.80 | 0.80 | 0.80 | 0.80 |
| Salicylic Acid | 0.50 | | | 0.50 | 0.50 | |
| Allantoin | | | | | | |
| Isopropyl Alcohol | | | 3.00 | | 3.00 | 3.00 |
| L(+)-Lactic Acid (88%) USP-ADM | 4.00 | 4.00 | 4.00 | | | |
| Benzyl Alcohol | | 1.00 | | 1.00 | | 1.00 |
| PEG-7-Glyceryl Cocoate (Cetiol HE) | 0.50 | 0.50 | 0.50 | 0.50 | 0.50 | 0.50 |
| EO/PO/EO Block Copolymer (Pluronic P105) | 0.50 | 0.50 | 0.50 | 0.50 | 0.50 | 0.50 |
| Sodium Dioctylsulfosuccinate (75%) | 0.15 | 0.15 | 0.15 | 0.15 | 0.15 | 0.15 |
| Polysorbate 80 (Tween 80) | 0.50 | 0.50 | 0.50 | 0.50 | 0.50 | 0.50 |
| Sodium Hydroxide (50%) | 1.25 | 1.00 | 1.00 | 0.25 | 0.25 | |
| Phosphoric acid* | | | | | | 0.50 |
| pH (Neat) | 3.49 | 3.49 | 3.49 | 3.56 | 3.58 | 3.49 |
| AOAC Germicidal Test 960.09 (20° C./30 Sec) | | | | | | |
| E. Coli (Log Reduction) | 7.90 | 7.90 | 1.00 | 4.43 | No Kill | No Kill |
| Staph. Aureus (Log Reduction) | 2.20 | 5.30 | No kill | 7.80 | 2.91 | 0.60 |

*Phosphoric Acid was used to adjust the pH of the formula; all formulae were adjusted to have a pH close to 3.50

Solutions containing three antimicrobial agents, as shown in Table 4, also showed efficacy toward at least one strain of bacteria. Excellent results against both Gram positive and Gram negative bacteria were observed with solutions 20 and 22 containing, respectively: (20) lactic acid, salicylic acid and benzyl alcohol; and (22) salicylic acid, benzyl alcohol and isopropyl alcohol.

TABLE 4
Kill Property Study: Selection of Germicidals:
Three Germicides Combined

| | Solution: | | | |
|---|---|---|---|---|
| | 20 | 21 | 22 | 23 |
| Antimicrobial Agents | | | | |
| Lactic Acid (88%)- 4.00% | + | + | | + |
| Salicylic Acid - 0.50% | + | | + | + |
| Benzyl Alcohol - 1.00% | + | + | + | |
| Isopropyl Alcohol - 3.00% | | + | + | + |
| Ingredients (wt %) | | | | |
| Water | 90.40 | 87.90 | 92.40 | 88.40 |
| Xanthan Gum (Novaxan D/ADM) | 0.40 | 0.40 | 0.40 | 0.40 |
| Polyvinylpyrrolidone K-30 | 0.80 | 0.80 | 0.80 | 0.80 |
| Salicylic Acid | 0.50 | | 0.50 | 0.50 |
| Allantoin | | | | |
| Isopropyl Alcohol | | 3.00 | 3.00 | 3.00 |
| L(+)-Lactic Acid (88%) USP-ADM | 4.00 | 4.00 | | 4.00 |
| Benzyl Alcohol | 1.00 | 1.00 | 1.00 | |
| PEG-7-Glyceryl Cocoate (Cetiol HE) | 0.50 | 0.50 | 0.50 | 0.50 |
| EO/PO/EO Block Copolymer | 0.50 | 0.50 | 0.50 | 0.50 |
| (Pluronic P105) | | | | |
| Sodium Dioctylsulfosuccinate(75%) | 0.15 | 0.15 | 0.15 | 0.15 |
| Polysorbate 80 (Tween 80) | 0.50 | 0.50 | 0.50 | 0.50 |
| Sodium Hydroxide (50%) | 1.25 | 1.25 | 0.25 | 1.25 |
| Phosphoric acid | | | | |
| pH (Neat) | 3.49 | 3.47 | 3.54 | 3.49 |
| AOAC Germicidal Test 960.09 | | | | |
| (20° C./30 Sec) | | | | |
| E. Coli (Log Reduction) | 7.90 | 7.90 | 7.90 | 4.67 |
| Staph. Aureus (Log Reduction) | 7.80 | 2.80 | 7.80 | 4.70 |

Solutions containing three antimicrobial agents, as shown in Tables 5 and 6, also showed efficacy toward at least one strain of bacteria. Excellent results against both Gram positive and Gram negative bacteria were observed with solutions 24-29 containing: lactic acid, salicylic acid and isopropyl alcohol with varying concentrations of salicylic acid from 0.50% to 1.00%. These compositions show excellent synergy between lactic acid, salicylic acid and isopropyl alcohol without benzyl alcohol in the compositions. Excellent results against both Gram positive and Gram negative bacteria were observed in Table 5 with solutions 30-31 containing: lactic acid, salicylic acid and benzyl alcohol with salicylic acid 0.50%. Excellent results were also obtained using each of the barrier formulations in Table 6.

TABLE 5
Kill Property Study: Selection of Germicidals: Three Germicides
Combined

| | Solution: | | | | | | | |
|---|---|---|---|---|---|---|---|---|
| | 24 | 25 | 26 | 27 | 28 | 29 | 30 | 31 |
| Antimicrobial Agents | | | | | | | | |
| Lactic Acid (88%) - 4.00% | + | + | + | + | + | + | + | + |
| Salicylic Acid - 0.50% | + | + | + | + | + | + | + | + |
| Benzyl Alcohol - 1.00% | | | | | | | + | + |
| Isopropyl Alcohol - 3.00% | + | + | + | + | + | + | | |
| Ingredients (wt %) | | | | | | | | |
| Water | 78.78 | 78.68 | 78.58 | 78.48 | 78.38 | 78.28 | 80.78 | 80.78 |
| Xanthan Gum (Novaxan | 0.40 | 0.40 | 0.40 | 0.40 | 0.40 | 0.40 | 0.40 | 0.40 |
| D/ADM) | | | | | | | | |
| Polyvinylpyrrolidone K-30 | 0.80 | 0.80 | 0.80 | 0.80 | 0.80 | 0.80 | 0.80 | 0.80 |
| Salicylic Acid | 0.50 | 0.60 | 0.70 | 0.80 | 0.90 | 1.00 | 0.50 | 0.50 |
| Allantoin | 0.10 | 0.10 | 0.10 | 0.10 | 0.10 | 0.10 | 0.10 | 0.10 |
| Isopropyl Alcohol | 3.00 | 3.00 | 3.00 | 3.00 | 3.00 | 3.00 | | |
| Glycerin | | | | | | | 10.00 | |
| Sorbitol 100% USP | 10.00 | 10.00 | 10.00 | 10.00 | 10.00 | 10.00 | | 10.00 |
| L(+)-Lactic Acid (88%) USP- | 4.00 | 4.00 | 4.00 | 4.00 | 4.00 | 4.00 | 4.00 | 4.00 |
| ADM | | | | | | | | |
| Benzyl Alcohol | | | | | | | 1.00 | 1.00 |
| EO/PO/EO Block Copolymer | | | | | | | 0.50 | |
| (Pluronic P105) | | | | | | | | |
| EO/PO/EO Block Copolymer | 0.50 | 0.50 | 0.50 | 0.50 | 0.50 | 0.50 | | 1.00 |
| (Pluronic F108) | | | | | | | | |
| Sodium | 0.15 | 0.15 | 0.15 | 0.15 | 0.15 | 0.15 | 0.15 | 0.15 |
| Dioctylsulfosuccinate (75%) | | | | | | | | |
| Polysorbate 80 (Tween 80) | 0.50 | 0.50 | 0.50 | 0.50 | 0.50 | 0.50 | 0.50 | |
| Sodium Hydroxide (50%) | 1.25 | 1.25 | 1.00 | 0.25 | 0.25 | 1.25 | 1.25 | 1.25 |
| FD&C Yellow 5 | 0.012 | 0.012 | 0.012 | 0.012 | 0.012 | 0.012 | 0.012 | 0.012 |
| FD&C Blue 1 | 0.008 | 0.008 | 0.008 | 0.008 | 0.008 | 0.008 | 0.008 | 0.008 |
| pH (Neat) | 3.49 | 3.47 | 3.49 | 3.54 | 3.46 | 3.48 | 3.54 | 3.50 |
| Viscosity LV2 30 rpm, cPS | 718 | 687 | 694 | 733 | 759 | 738 | 665 | 695 |
| AOAC Germicidal Test 960.09 | | | | | | | | |
| (20° C./30 Sec) | | | | | | | | |
| E. Coli (Log Reduction) | 7.92 | 8.50 | 8.50 | 6.10 | 8.50 | 8.50 | 8.04 | 7.95 |
| Staph. Aureus (Log Reduction) | 8.50 | 8.50 | 8.50 | 6.10 | 8.50 | 8.50 | 7.85 | 7.04 |

TABLE 6
Kill Property Study: Selection of Germicidals: Three Germicides
Combined- Barrier Formulations

| | Solution | | | |
|---|---|---|---|---|
| | 32 | 33 | 34 | 35 |
| Ingredients (wt %) | | | | |
| Water | 80.28 | 80.28 | 80.28 | 80.28 |
| Xanthan Gum (Kelco | 0.40 | 0.40 | 0.40 | 0.40 |
| Keltrol RD/Kelco) | | | | |
| Polyvinylpyrrolidone | 0.40 | 0.40 | 0.80 | 0.80 |
| K-30 | | | | |
| Salicylic Acid | 0.50 | 0.50 | 0.50 | 0.50 |
| Allantoin | 0.10 | 0.10 | 0.10 | 0.10 |
| FD&C Yellow 5-E102 | 0.0114 | 0.0114 | 0.0114 | 0.0114 |
| FD&C Blue 1-E133 | 0.0086 | 0.0086 | 0.0086 | 0.0086 |
| Glycerine | 10.00 | 10.00 | 10.00 | 10.00 |
| L(+)-Lactic Acid | 4.00 | 4.00 | 4.00 | 4.00 |
| (88%) USP-ADM | | | | |
| Benzyl Alcohol | 1.00 | 1.00 | 1.00 | 1.00 |
| PEG-7-Glyceryl Cocoate | 0.50 | 0.50 | 0.50 | 0.50 |
| (Cetiol HE) | | | | |
| EO/PO/EO Block | 0.50 | 0.50 | 0.50 | 0.50 |
| Copolymer Pluronic | | | | |
| P105 | | | | |
| Sodium | 0.15 | 0.15 | 0.15 | 0.15 |
| Dioctylsulfosuccinate | | | | |
| (75%) | | | | |
| Polysorbate 80 | 0.50 | 0.50 | 0.50 | 0.50 |
| (Tween 80) | | | | |
| Sodium Hydroxide | 1.25 | 1.25 | 1.25 | 1.50 |
| (50%) | | | | |
| pH (Neat) | 3.510 | 3.510 | 3.500 | 3.8 |
| Viscosity LV2 30 rpm | 616 | 580 | 580 | 635 |
| AOAC Germicidal | | | | |
| Test 960.09 | | | | |
| (20° C./30 Sec) | | | | |
| E. Coli (Log Reduction) | 7.5 | 7.50 | 8 | 8 |
| Staph. Aureus | 7.3 | 6.40 | 6.6 | 5.2 |
| (Log Reduction) | | | | |

Table 7 shows that good to excellent results were obtained against both Gram positive and Gram negative bacteria using four components.

TABLE 7
Kill Property Study: Selection of Germicidals:
Four Germicides Combined

| | Solution: |
|---|---|
| | 36 |
| Antimicrobial Agents | |
| Lactic Acid (88%)- 4.00% | + |
| Salicylic Acid - 0.50% | + |
| Benzyl Alcohol - 1.00% | + |
| Isopropyl Alcohol - 3.00% | + |
| Ingredients (wt %) | |
| Water | 87.40 |
| Xanthan Gum (Novaxan D/ADM) | 0.40 |
| Polyvinylpyrrolidone K-30 | 0.80 |
| Salicylic Acid | 0.50 |
| Allantoin | |
| Isopropyl Alcohol | 3.00 |
| L(+)-Lactic Acid (88%) USP-ADM | 4.00 |
| Benzyl Alcohol | 1.00 |
| PEG-7-Glyceryl Cocoate (Cetiol HE) | 0.50 |
| EO/PO/EO Block Coploymer (Pluronic P105) | 0.50 |
| Sodium Dioctylsulfosuccinate(75%) | 0.15 |
| Polysorbate 80 (Tween 80) | 0.50 |
| Sodium Hydroxide (50%) | 1.25 |
| pH (Neat) | 3.5 |
| AOAC Germicidal Test 960.09 (20° C./30 Sec) | |
| E. Coli (Log Reduction) | 8.00 |
| Staph. Aureus (Log Reduction) | 7.80 |

The synergy observed when at least two antimicrobial agents are combined is novel and surprising. Results are particularly good when benzyl alcohol is combined with protic organic acid(s) such as lactic acid and/or salicylic acid. Results are also particularly good when isopropyl alcohol is combined with protic organic acids such as lactic acid and salicylic acid.

Tables 8 and 9 list suitable ingredients that may be combined in biocidal compositions as described above. It will be appreciated that the compositions may be applied as dips or sprays having utility as teat dips. It will be further appreciated that the compositions may be formulated as non-barrier or barrier dips. Furthermore, the compositions may be formulated in various strengths and used in a variety of applications, such as the cleaning of milk processing apparatus. The preferred ranges shown are for teat dip formulations.

TABLE 8
Barrier Teat Dip Compositions-Preferred Ranges of Ingredients

| | | Broadly Preferred | Preferred | More Preferred | Still More Preferred |
|---|---|---|---|---|---|
| Functional Ingredients | | Range (% w/w) | Range (% w/w) | Range (% w/w) | Range (% w/w) |
| Germicidal Agent(s) and Mixture thereof | | 0.40-30.00 | 0.60-25.00 | 0.80-20.00 | 1.00-15.00 |
| (Organic Acids) | L(+)-Lactic Acid* | 0.02-20.00 | 0.03-15.00 | 0.04-10.00 | 0.05-8.00 |
| | Salicylic Acid* | 0.02-20.00 | 0.03-15.00 | 0.04-10.00 | 0.05-8.00 |
| | Glycolic Acid | 0.02-20.00 | 0.03-15.00 | 0.04-10.00 | 0.05-8.00 |
| (Alcohols) | Benzyl Alcohol | 0.02-20.00 | 0.03-15.00 | 0.04-10.00 | 0.05-8.00 |
| | Isopropyl Alcohol | 0.02-20.00 | 0.03-15.00 | 0.04-10.00 | 0.05-8.00 |
| | n-Propyl Alcohol | 0.02-20.00 | 0.03-15.00 | 0.04-15.00 | 0.05-8.00 |
| | Ethyl Alcohol | 0.02-20.00 | 0.03-15.00 | 0.04-10.00 | 0.05-8.00 |
| | Phenyl Alkyl Alcohol (Phenyl Alkanol) | | | | |

| | | | | |
|---|---|---|---|---|
| Ratio of Organic Acid to Alcohol | 1:0.001 to | 1:0.002 to | 1:0.004 to | 1:0.006 to |
| | 1:1000 | 1:500 | 1:250 | 1:160 |
| Film Forming/Barrier Forming Agent(s) and Mixture | 0-15.00 | 0.30-13.00 | 0.40-11.00 | 0.50-10.00 |
| thereof | | | | |
| Polyvinyl Pyrrolidone (PVP) | 0-13.00 | 0.03-12.00 | 0.04-10.00 | 0.05-8.00 |
| Cellulose, Carboxyalkyl and Hydroxyalkyl Cellulose | 0-13.00 | 0.03-12.00 | 0.04-10.00 | 0.05-8.00 |
| Polyethylene Glycol (Varying Degree of Mol. Wt.) | 0-13.00 | 0.03-12.00 | 0.04-10.00 | 0.05-8.00 |
| Polyvinyl Alcohol (PVA-Varying Degree of Mol. Wt.) | 0-13.00 | 0.03-12.00 | 0.04-10.00 | 0.05-8.00 |
| Polylactic Acid/Alkali Polylactate (Various Mol. Wt.) | 0-13.00 | 0.03-12.00 | 0.04-10.00 | 0.05-8.00 |
| EO/PO/EO Block Copolymer Pluronic P105 | 0-13.00 | 0.03-12.00 | 0.04-10.00 | 0.05-8.00 |
| EO/PO/EO Block Copolymer Pluronic F108 | 0-13.00 | 0.03-12.00 | 0.04-10 00 | 0.05-8.00 |
| Polysulfonic Acid, Polyalkyl Sulfonic Acid and AlkaliSalts, | 0-13.00 | 0.03-12.00 | 0.04-10.00 | 0.05-8.00 |
| Polyacrylamide | | | | |
| Sodium Alginate, Sodium Alginate with Calcium Ion | 0-13.00 | 0.03-12.00 | 0.04-10.00 | 0.05-8.00 |
| PEG-8-40, PEG-4-80 Stearate, PEG-4-80 Hydroxy Stearate | 0-13.00 | 0.03-12.00 | 0.04-10.00 | 0.05-8.00 |
| Sorbitan Monostearate | 0-13.00 | 0.03-12.00 | 0.04-10.00 | 0.05-8.00 |
| PEG-15-Hydroxystearate | 0-13.00 | 0.03-12.00 | 0.04-10.00 | 0.05-8.00 |
| Thickeners/Viscosity Modifier(s) and Mixture thereof | 0-15.00 | 0.30-13.00 | 0.40-11.00 | 0.50-10.00 |
| Xanthan Gum | 0-13.00 | 0.03-11.00 | 0.04-10.00 | 0.05-8.00 |
| Cellulose and Hydroxyalkyl Cellulose Derivatives | 0-13.00 | 0.03-11.00 | 0.04-10.00 | 0.05-8.00 |
| High Molecular weight Cross-linked | 0-13.00 | 0.03-11.00 | 0.04-10.00 | 0.05-8.00 |
| Polyacrylates(Carbopol) | | | | |
| Chitosan and Chitosan Derivatives | 0-13.00 | 0.03-11.00 | 0.04-10.00 | 0.05-8.00 |
| Guar Gum | 0-13.00 | 0.03-11.00 | 0.04-10.00 | 0.05-8.00 |
| Sodium Alginate, Sodium Alginate with Calcium Salt, | 0-13.00 | 0.03-11.00 | 0.04-10.00 | 0.05-8.00 |
| Carrageenan Gum | | | | |
| Whey | 0-13.00 | 0.03-11.00 | 0.04-10.00 | 0.05-8.00 |
| Dextran Sulfate | 0-13.00 | 0.03-11.00 | 0.04-10.00 | 0.05-8.00 |
| Gelatin | 0-13.00 | 0.03-11.00 | 0.04-10.00 | 0.05-8.00 |
| Emollients and Skin Conditioning Agent(s) and Mixture | 0-30.00 | 2.00-25.00 | 3.00-20.00 | 4.00-15.00 |
| thereof | | | | |
| Glycerin | 0-20.00 | 0.03-15.00 | 0.04-13.00 | 0.05-10.00 |
| Sorbitol | 0-20.00 | 0.03-15.00 | 0.04-13.00 | 0.05-10.00 |
| Polyethylene Glycol (Varying Degree of Mol. Wt.) | 0-20.00 | 0.03-15.00 | 0.04-13.00 | 0.05-10.00 |
| Stearic Acid | 0-20.00 | 0.03-15.00 | 0.04-13.00 | 0.05-10.00 |
| Propylene Glycol | 0-20.00 | 0.03-15.00 | 0.04-13.00 | 0.05-10.00 |
| Monoglyceryl Fatty Alkanoate | 0-20.00 | 0.03-15.00 | 0.04-13.00 | 0.05-10.00 |
| PEG-7 Glycerol Cocoate | 0-20.00 | 0.03-15.00 | 0.04-13.00 | 0.05-10.00 |
| Fatty Alkyl Polyglucoside & Glyceryl Laurate/Cocoate) | 0-20.00 | 0.03-15.00 | 0.04-13.00 | 0.05-10.00 |
| Lanolin and Alkoxylated Lanolin | 0-20.00 | 0.03-15.00 | 0.04-13.00 | 0.05-10.00 |
| Allantoin | 0-20.00 | 0.03-15.00 | 0.04-13.00 | 0.05-10.00 |
| B₅ Provitamin | 0-20.00 | 0.03-15.00 | 0.04-13.00 | 0.05-10.00 |
| Guar Hydroxypropyltrimonium Chloride | 0-20.00 | 0.03-15.00 | 0.04-13.00 | 0.05-10.00 |
| Hydrolysed Silk Peptide/Silk Protein | 0-20.00 | 0.03-15.00 | 0.04-13.00 | 0.05-10.00 |
| Polysorbate 80 (Tween 80) | 0-20.00 | 0.03-15.00 | 0.04-13.00 | 0.05-10.00 |
| Polyquaternium-7 | 0-20.00 | 0.03-15.00 | 0.04-13.00 | 0.05-10.00 |
| Aloe Vera Gel | 0-20.00 | 0.03-15.00 | 0.04-13.00 | 0.05-10.00 |
| Surfactants and Wetting Agent and Mixture thereof | 0-20.00 | 0.20-15.00 | 0.30-10.00 | 0.50-8.00 |
| Fatty Alkyl Polyglucoside (Varying Degree of Mol. Wt.) | 0-15.00 | 0.03-10.00 | 0.04-8.00 | 0.05-6.00 |
| Sodium Dioctyl Sulfosuccinate | 0-15.00 | 0.03-10.00 | 0.04-8.00 | 0.05-6.00 |
| Sodium Cocoyl Isethionate | 0-15.00 | 0.03-10.00 | 0.04-8.00 | 0.05-6.00 |
| Sodium Cocoamidopropyl Betaine | 0-15.00 | 0.03-10.00 | 0.04-8.00 | 0.05-6.00 |
| Sodium α-Olefin (C14-16) Sulfate | 0-15.00 | 0.03-10.00 | 0.04-8.00 | 0.05-6.00 |
| Buffering and pH Controlling Agents and Mixture thereof | 0-12.00 | 0.20-10.00 | 0.30-8.00 | 0.40-5.00 |
| Citric Acid | 0-10.00 | 0.03-8.00 | 0.04-6.00 | 0.05-4.00 |
| Phosphoric Acid | 0-10.00 | 0.03-8.00 | 0.04-6.00 | 0.05-4.00 |
| Sodium Carbonate | 0-10.00 | 0.03-8.00 | 0.04-6.00 | 0.05-4.00 |
| Sodium Bicarbonate | 0-10.00 | 0.03-8.00 | 0.04-6.00 | 0.05-4.00 |
| Sodium Hydroxide | 0-10.00 | 0.03-8.00 | 0.04-6.00 | 0.05-4.00 |
| Dyes, Coloring and Aesthetic Agents and Mixture thereof | 0-5.00 | 0.0020-4.00 | 0.0030-3.00 | 0.0040-2.00 |
| FD&C Blue 1 | 0-4.00 | 0.0010-3.00 | 0.0015-2.00 | 0.002-1..00 |
| FD&C Yellow 5 | 0-4.00 | 0.0010-3.00 | 0.0015-2.00 | 0.002-1..00 |
| FD&C Red 3 | 0-4.00 | 0.0010-3.00 | 0.0015-2.00 | 0.002-1..00 |
| FD&C Red 4 | 0-4.00 | 0.0010-3.00 | 0.0015-2.00 | 0.002-1..00 |
| FD&C Red 40 | 0-4.00 | 0.0010-3.00 | 0.0015-2.00 | 0.002-1..00 |

| Physical Property | Broad | Preferred | Most Preferred | Still Most Preferred |
|---|---|---|---|---|
| pH (Neat) | 2.50-7.00 | 3.00-6.50 | 3.20-6.00 | 3.25-5.00 |
| Viscosity, cPs (Dynamic; Brookfield DV-II, 30 rpm, spindle # | 100-3,000 | 200-2,500 | 250-2000 | 300-1600 |
| LV2) | | | | |

*Note:
Additional organic or equivalent acids may be used in place of lactic and/or salicylic acid including, for example, ascorbic acid, tartaric acid, citric acid, glycolic acid, maleic acid, succinic acid, mandelic acid, dodecylbenzenesulfonic acid, propionic acid, hexanoic acid, octanoic acid, nonanoic acid, decanoic acid, undecanoic acid, gluconic acid, malic acid, benzoic acid, aspartic acid, acetic acid, oxalic acid, glutamic acid, sulfamic acid, and adipic acid.

TABLE 9
Preferred Physical Properties For Teat Dip Use

| Physical | | | Most | Still |
|---|---|---|---|---|
| Property | Broad | Preferred | Preferred | Most Preferred |
| pH (Neat) | 2.50-7.00 | 3.00-6.50 | 3.20-6.00 | 3.25-5.00 |
| Viscosity, | 1-3,000 | 200-2,500 | 250-2,000 | 300-1,600 |
| cPs | | | | |
| (Dynamic; | | | | |
| Brookfield | | | | |
| DV-II, | | | | |
| 30 rpm, | | | | |
| spindle | | | | |
| # LV2) | | | | |

TABLE 10
Preferred Physical Properties For Sprayable Teat Dip and Low
Viscosity Hard Surface Care Use

| Physical | Broadly | | Most | Still Most |
|---|---|---|---|---|
| Property | Preferred | Preferred | Preferred | Preferred |
| pH (Neat) | 0.5-7.00 | 1.00-6.50 | 2.00-6.00 | 2.5-5.00 |
| Viscosity, cPs (Dynamic; | 1-200 | 1-100 | 1-50 | 1-25 |
| Brookfield DV-II, 30 | | | | |
| rpm, spindle | | | | |
| # LV2) | | | | |

TABLE 11
Preferred Physical Properties For Gel Type products (Thixotropic,
Pseudoplastic or Viscoelastic) for Teat Dip, Hand Soap, Surgical
Scrub, Hand Sanitizer, Personal Care and Hard Surface Care Use

| | | | | Still |
|---|---|---|---|---|
| | Broadly | | Most | Most |
| Physical Property | Preferred | Preferred | Preferred | Preferred |
| pH (Neat) | 0.50-7.00 | 1.00-6.50 | 2.00-6.00 | 2.5- |
| | | | | 5.00 |
| Viscosity, cPs (Dynamic; | 100- | 200- | 250- | 300- |
| Brookfield DV-II, 20 | 20,000 | 15,000 | 13,000 | 10,000 |
| rpm, spindle # 4) | | | | |

Table 8 shows that a broadly preferred range of germicidal or antimicrobial active agents, in combination and in a state that is made ready for use, is from 0.4% to 30% by weight of the antimicrobial solution. These may be used individually in ranges from 0.02% to 20% (w/w) to achieve at least some mutual benefit. This means that the ratio of organic acid to alcohol may range from 1:0.001 to 1:1000 by weight. The still more preferred range is from 0.05% to 8% (w/w), which equates to a ratio of from 1:0.006 to 1:160. The most preferred range is from 0.5 to 4 by weight. Although the use of lactic acid and/or salicylic acid is particularly preferred, other organic acids may be substituted for or used in addition to lactic acid and/or salicylic acid.

The antimicrobial compositions may be sold as concentrates that contain ingredients in excess of what is reported in Table 8, but may be diluted to values within these ranges by the addition of a carrier, such as water, to make the compositions ready for use. By way of example, in some embodiments the concentrated solutions may be provided within the ratios shown and diluted to achieve the range values as specified in Table 8. Thus, for example, the concentrates may be mixed with a diluent 1×, 2×, 3×, 4×, 5× or even up to 20× or more. A preferred diluent is water.

The barrier or film/forming agents of Table 8 may be used to provide barrier protection, for example, as a barrier teat dip or wearable hand sanitizer. A broadly preferred range of these materials in combination is from 0.2% to 15% by weight of the antimicrobial composition. This may be achieved by combining individual ingredients in ranges from 0.02% to 13% (w/w). In a still more preferred sense, the individual ingredients are present in combination at from 0.5% to 10% (w/w), and individually at from 0.05% to 8% (w/w).

The thickeners/viscosity modifiers of Table 8 may be used to adjust solution viscosity, for example, to formulate the consistency of the antimicrobial compositions according to an intended environment of use. In a particularly preferred example, an optimized solution viscosity imparts vertical clinging of the product onto substrate and minimize dripping of the product to avoid waste. This is particularly important in such vertical surface applications such as teat tipping of cows.

The emollients/skin conditioning agents of Table 8 may be used to condition skin, for example, to provide a lotion with a soothing effect to the hands or to condition the teats and udders of a dairy animal. A broadly preferred range of these materials in combination is from 1% to 30% by weight of the antimicrobial composition. This may be achieved by combining individual ingredients in ranges from 0.02% to 20% (w/w). In a still more preferred sense, the individual ingredients are present in combination at from 4% to 15% (w/w), and individually at from 0.05% to 10% (w/w).

As shown in Table 9, a teat dip formulation in the state of being made ready for use may have a preferred dynamic viscosity ranging from 100 cPs to 3000 cPs at room temperature. A viscosity ranging from 1 cPs to 300 cPs may be more suitable for some embodiments of a hard surface disinfectant. A viscosity ranging from 25 cPs to 300 cPs may be more suitable for use as a surgical scrub or hand sanitizer. These objectives may be achieved by combining viscosity modifiers in combination at 0.2 to 15% (w/w) and individual ingredients in ranges from 0.02% to 13% (w/w) on the basis of total composition weight. In a still more preferred sense, the individual ingredients are present in combination at from 0.5% to 10% (w/w), and individually at from 0.05% to 8% (w/w). It will be appreciated that some ingredients, such as cellulose, xanthan gum have a dual functionality as barrier or film-forming agents and viscosity modifiers, and that other solution ingredients may operate to increase or decrease viscosity.

Tables 10 and 11 show properties of other formulations that may be used, for example, as a sprayable teat dip, and gel-type products (thixotropic, pseudoplastic or viscoelastic or gel products) for teat dips, hand soaps, surgical scrubs, hand sanitizers, personal care products and hard surface care products.

Those skilled in the art will appreciate that the foregoing discussion teaches by way of example, and not by limitation. Insubstantial changes may be imposed upon the specific embodiments that are shown and described without departing from the scope and spirit of the invention.

We claim:

1. An antimicrobial liquid composition comprising enhanced antimicrobial effective amounts of at least two antimicrobial agents selected from the group consisting of an organic acid and an alcohol, the alcohol selected from the group consisting of benzyl alcohol, a low molecular weight alcohol having a carbon number less than five, and combinations thereof.

2. The antimicrobial composition of claim 1, wherein the alcohol includes the low molecular weight alcohol having a carbon number less than five.

3. The antimicrobial composition of claim 1, wherein the low molecular weight alcohol is selected from the group consisting of ethanol, isopropanol, n-propanol and combinations thereof.

4. The antimicrobial composition of claim 1, wherein the alcohol includes benzyl alcohol.

5. The antimicrobial composition of claim 1, wherein the alcohol includes a combination of benzyl alcohol and the low molecular weight alcohol.

6. The antimicrobial composition of claim 1, wherein the organic acid is selected from the group consisting of lactic acid, salicylic acid, tartaric acid, citric acid, glycolic acid, maleic acid, ascorbic acid, succinic acid, mandelic acid, dodecylbenzenesulfonic acid, propionic acid, hexanoic acid, octanoic acid, nonanoic acid, decanoic acid, undecanoic acid, gluconic acid, malic acid, benzoic acid, aspartic acid, acetic acid, oxalic acid, glutamic acid, adipic acid, and combinations thereof.

7. The antimicrobial composition of claim 1, wherein the organic acid is selected from the group consisting of lactic acid, salicylic acid, and combinations thereof.

8. The antimicrobial composition of claim 1 further comprising a pharmaceutical carrier.

9. The antimicrobial composition of claim 8, wherein the pharmaceutical carrier is predominantly water.

10. The antimicrobial composition of claim 1 further comprising an additive.

11. The antimicrobial composition of claim 10, wherein the additive is a viscosity controlling agent.

12. The antimicrobial composition of claim 11, wherein the viscosity controlling agent is a material selected from hemicellulose, plant gum, cellulose and derivatives thereof; starch and derivatives thereof; microbial polysaccharides, plant polysaccharides, sea-weed polysaccharides; and combinations thereof.

13. The antimicrobial composition of claim 12, wherein the cellulose derivative is selected from the group consisting of cellulose, methyl cellulose, ethyl cellulose, hydroxyethylcellulose, hydroxymethyl cellulose, carboxyethyl cellulose, and carboxymethyl cellulose.

14. The antimicrobial composition of claim 12, wherein the microbial polysaccharides, plant polysaccharides and sea-weed polysaccharides is selected from the group consisting guar gum, xanthan gum, sodium alginate, sodium alginate cross-linked with calcium salt, carrageenan; and combinations thereof.

15. The antimicrobial composition of claim 1, wherein the enhanced antimicrobial effective amounts include the organic acid and the alcohol each being present in respective amounts of at least 0.02% by total weight of the antimicrobial composition.

16. The antimicrobial composition of claim 1 further comprising a barrier and film forming agent.

17. The antimicrobial composition of claim 16, wherein the barrier and film forming agent is selected from latex, arabinoxylanes, glucomannanes, guar gum, gum arabic, johannistree gums, cellulose, methyl cellulose, ethyl cellulose, hydroxymethyl cellulose, hydroxyethyl cellulose, carboxymethyl cellulose, carboxyethyl cellulose starch, hydroxyethyl starch, xanthan gum, curdlan, pullulan, dextran, chitosan, glycerol, sodium alginate, sodium alginate cross-linked with calcium salt, carrageenan, ethyleneoxide/propylene oxide/ethyleneoxide block copolymers, high molecular weight polyacrylate, high molecular weight cross-linked polyacrylate, carbomer, poly(vinyl alcohol) (PVA), and poly(N-vinylpyrrolidone) (PVP) and combinations there of.

18. The antimicrobial composition of claim 1 further comprising an opacifying agent.

19. The antimicrobial composition of claim 1, wherein the antimicrobial agents are present in combination at a concentration ranging from 0.40% to 30% by total weight of the antimicrobial composition.

20. The antimicrobial composition of claim 1, wherein the antimicrobial agents are each present in an amount ranging from 0.02% to 20% by total weight of the antimicrobial composition.

21. The antimicrobial composition claim 20, further comprising from 0.01% to 15% (w/w) of a viscosity modifier.

22. The antimicrobial composition of claim 21, further comprising from 0.001 % to 5.00% w/w of a coloring agent.

23. The antimicrobial composition of claim 21, further comprising from 0.20% to 15% (w/w) of a barrier-forming agent.

24. The antimicrobial composition of claim 20, further comprising from 1 % to 30% (w/w) of emollients and skin conditioning agents.

25. The antimicrobial composition of claim 24 having a pH ranging from 2.5 to 7.0.

26. The antimicrobial composition of claim 25 having a dynamic viscosity ranging from 100 cPs to 3000 cPs.

27. The antimicrobial composition claim 25 having a dynamic viscosity ranging from 1 cPs to 300 cPs.

28. The antimicrobial composition of claim 1, wherein the enhanced effective amounts include a ratio of the organic acid to the alcohol ranging from 1:0.001 to 1:1000 on a weight percent basis.

29. The antimicrobial composition of claim 1 in concentrated form such that the composition retains antimicrobial effective amounts when diluted by a volume of diluent not less than an equal volume of the antimicrobial composition in concentrated form.

30. A method for the prophylactic treatment of a lactating animal against mastitis comprising applying to the teats of a lactating animal in need of the prophylactic treatment a composition comprising the antimicrobial composition of claim 1.

31. The method of claim 30, wherein the lactating animal is a cow.

32. The method of claim 30, wherein the antimicrobial agents are present in amounts that result in a prophylactic effect against mastitis when the composition is applied topically.

33. The method of claim 30, wherein the composition is applied to the teats of the lactating animal by dipping or spraying.

34. A method for the mitigation of bacteria on a hard surface comprising a step of applying to the hard surface a composition comprising the antimicrobial composition of claim 1.

35. A method for the mitigation of bacteria on a human hand comprising a step of applying to the human hand a composition comprising the antimicrobial composition of claim 1.

36. The method of claim 35, wherein the step of applying is performed in a manner consistent with preparation for surgery, and further including a step of performing surgery.

37. The method of claim 35, wherein the step of applying is performed in a manner consistent with personal care.

38. A method for controlling bovine mastitis comprising contacting the teats of a cow with a substantially nonirritating teat dip that contains enhanced antimicrobial effective amounts of
an organic acid selected from the group consisting of lactic acid, salicylic acid, and combinations thereof, and
an alcohol selected from the group consisting of benzyl alcohol, phenyl alkyl alcohol (phenyl alkanol), ethyl alcohol, n-propyl alcohol, isopropyl alcohol, and combinations thereof
in a pharmaceutically acceptable carrier, wherein the amount of each antimicrobial agent present in the teat dip ranges from 0.02% to 20% (w/w) based on the total weight of the teat dip.

39. The method of claim 38, wherein the alcohol that is used in the step of contacting comprises n-propyl alcohol.

40. The method of claim 38, wherein the alcohol that is used in the step of contacting comprises isopropyl alcohol.

41. The method of claim 38, wherein the alcohol that is used in the step of contacting comprises ethyl alcohol.

42. The method of claim 38, wherein the alcohol that is used in the step of contacting comprises benzyl alcohol.

43. The method of claim 38, wherein the alcohol that is used in the step of contacting comprises phenyl alkyl alcohol (phenyl alkanol)

44. The method of claim 43, wherein the alcohol that is used in the step of contacting comprises phenyl alkyl alcohol (phenyl alkanol), wherein alkyl group is selected from methyl, ethyl, isopropyl, n-propyl, n-butyl, isobutyl, ethyledene, propyledene moiety.

45. The method of claim 38, wherein the alcohol that is used in the step of contacting comprises a combination of propyl alcohol and benzyl alcohol.

46. A veterinary method for the mitigation of bacterial on a hoof comprising a step of applying to the hoof a composition comprising the antimicrobial composition of claim 1.
